(19) United States
(12) Patent Application Publication
(10) Pub. No.: US 2023/0309806 A1
(43) Pub. Date: Oct. 5, 2023

(54) ENDOSCOPE IMAGE PICKUP DEVICE AND ENDOSCOPE

(71) Applicant: FUJIFILM Corporation, Tokyo (JP)

(72) Inventors: Kazuaki TAKAHASHI, Kanagawa (JP); Takuro ASAOKA, Kanagawa (JP)

(73) Assignee: FUJIFILM Corporation, Tokyo (JP)

(21) Appl. No.: 18/188,285

(22) Filed: Mar. 22, 2023

(30) Foreign Application Priority Data
Mar. 31, 2022 (JP) 2022-059241

(51) Int. Cl. A61B 1/05; A61B 1/00

(52) U.S. Cl. CPC A61B 1/05; A61B 1/00096; A61B 1/00163

(57) ABSTRACT

Provided are an endoscope image pickup device and an endoscope capable of realizing a firm fixation and a stable connection strength.

There is provided an endoscope image pickup device that acquires an image of an observation target, the endoscope image pickup device including: a lens barrel provided with an internal image pickup lens; an image pickup element that receives light which has passed through the image pickup lens to perform photoelectric conversion; a holder that holds the lens barrel; a signal cable electrically connected to the image pickup element; and a connecting member that connects the holder and the signal cable to each other. The holder and the connecting member are engaged with each other at an engaging portion, and the engaging portion is provided with an adhesive layer containing a filler.

### CROSS-REFERENCE TO RELATED APPLICATIONS

The present application claims priority under 35 U.S.C. § 119 to Japanese Patent Application No. 2022-059241, filed on Mar. 31, 2022. The above application is hereby expressly incorporated by reference, in its entirety, into the present application.

### BACKGROUND OF THE INVENTION

### 1. Field of the Invention

The present invention relates to an endoscope image pickup device and an endoscope that acquire an image of an observation target, and in particular, to an endoscope image pickup device and an endoscope using a filler-containing adhesive.

### 2. Description of the Related Art

In recent years, a diagnosis and the like using an endoscope system that comprises a light source device for an endoscope, an endoscope (endoscope scope), and a processor device have been widely performed.

An insertion part to be inserted into a body of a person to be examined is provided, and illumination light from the light source device for an endoscope passes through the insertion part and is emitted to an observation target. The endoscope picks up an image of the observation target irradiated with illumination light with an image pickup element to generate an image signal. The processor device performs image processing on the image signal generated by the endoscope to generate an observation image to be displayed on a monitor. The image pickup element is electrically connected to a signal cable via a circuit board formed of a flexible wiring board or the like, and the signal cable is electrically connected to the processor device.

Although the image pickup element, a lens barrel, and the like are provided at a distal end of the insertion part, the distal end of the insertion part is also be bent in conformity with various types of imaging in order for the distal end of the insertion part to realize the various types of imaging in a case in which the endoscope is used. In this case, since the distal end of the insertion part to be inserted into the body is bent in a state in which the image pickup element, the lens barrel, and the like are provided as described above, a force is applied to a joining part of each member, which may cause damage, such as separation of the joining part. An endoscope having a firm joint has been proposed in order to suppress damage, such as separation.

For example, an endoscope of JP2016-158769A comprises a solid-state image pickup element that photoelectrically converts an optical image formed via an imaging lens, a circuit board that is electrically connected to the solid-state image pickup element, a signal cable that is electrically connected to the circuit board, an optical member holding portion that holds the imaging lens or a prism, a connecting member of which one end is stuck to the signal cable, of which the other end is provided with a locking claw that is locked to the optical member holding portion, and which connects the optical member holding portion and the signal cable to each other, and a mounting member that is mounted on at least a part of an outer periphery of the optical member holding portion. The mounting member is in contact with the locking claw locked to the optical member holding portion and sandwiches at least a part of the locking claw with the optical member holding portion.

### SUMMARY OF THE INVENTION

In JP2016-158769A described above, the locking claw is used for fixing the optical member holding portion and the connecting member. Since JP2016-158769A has a configuration in which the locking claw is hooked, there is a limit to a firm fixation. In addition, in the configuration in which the locking claw is hooked, the degree of hooking of the locking claw affects a connection strength. In a case in which the hooking of the locking claw becomes insufficient because of repeated use of the endoscope, the connection strength may be weakened. As described above, it is difficult to realize the firm fixation and the stable connection strength only with the locking claw.

An object of the present invention is to provide an endoscope image pickup device and an endoscope capable of realizing a firm fixation and a stable connection strength.

In order to achieve the above-described object, according to an aspect of the present invention, there is provided an endoscope image pickup device that acquires an image of an observation target, the endoscope image pickup device comprising: a lens barrel provided with an internal image pickup lens; an image pickup element that receives light which has passed through the image pickup lens to perform photoelectric conversion; a holder that holds the lens barrel; a signal cable electrically connected to the image pickup element; and a connecting member that connects the holder and the signal cable to each other, in which the holder and the connecting member are engaged with each other at an engaging portion, and the engaging portion is provided with an adhesive layer containing a filler.

It is preferable that the adhesive layer has a thickness of 5 μm or more.

It is preferable that the connecting member has a pair of arm portions facing each other, the holder has a projecting portion on a side surface, and the projecting portion protrudes in a direction perpendicular to the side surface with respect to the arm portion in a state in which the arm portion is bonded to the side surface of the holder via the adhesive layer.

It is preferable that the filler has a form of particles, and a height of the projecting portion is larger than an average particle diameter of the filler.

It is preferable that the filler has a form of particles, the projecting portions are disposed on the side surface of the holder so as to face each other at an interval along a height direction orthogonal to an optical axis of the image pickup lens, the arm portion is disposed between the projecting portions, and a gap between the projecting portion and the arm portion in the height direction is wider than an average particle diameter of the filler.

It is preferable that the arm portion has a latch portion that latches onto the projecting portion of the holder in an optical axis direction of an optical axis of the image pickup lens, and

that a position of the connecting member in the optical axis direction is restricted by the latch portion.

It is preferable that a distal end portion of each of the pair of arm portions is bent inward, the holder has a groove that is engaged with the distal end portion of the arm portion, and the groove is provided with the adhesive layer in a state in which the distal end portion is locked to the groove.

It is preferable that the connecting member has a pair of arm portions facing each other and has an opening portion provided at a distal end of the arm portion, the holder has engaging projecting portions disposed so as to face each other in a disposition direction of the arm portions, and the engaging projecting portion has a polygonal outer shape, the opening portion of the arm portion is engaged with the engaging projecting portion of the holder, and the opening portion surrounds at least three side surfaces of the engaging projecting portion, and there is a gap between the engaging projecting portion and the opening portion, the filler has a form of particles, and the gap is wider than an average particle diameter of the filler.

It is preferable that the adhesive layer has the filler and a resin containing the filler, and a thermal conductivity of the filler is higher than a thermal conductivity of the resin.

In addition, according to an aspect of the present invention, there is provided an endoscope comprising: the endoscope image pickup device of the aspect of the present invention.

According to the present invention, it is possible to provide an endoscope image pickup device and an endoscope capable of realizing a firm fixation and a stable connection strength.

### BRIEF DESCRIPTION OF THE DRAWINGS

FIG. 1 is a schematic diagram showing an example of an endoscope system of an embodiment of the present invention.

FIG. 2 is a schematic perspective view showing a first example of an endoscope image pickup device of the embodiment of the present invention.

FIG. 3 is a schematic side view showing the first example of the endoscope image pickup device of the embodiment of the present invention.

FIG. 4 is a schematic enlarged side view showing a part of the first example of the endoscope image pickup device of the embodiment of the present invention.

FIG. 5 is a schematic cross-sectional view taken along line A-A of the first example of the endoscope image pickup device of the embodiment of the present invention.

FIG. 6 is a schematic enlarged plan view showing a part of the first example of the endoscope image pickup device of the embodiment of the present invention.

FIG. 7 is a schematic perspective view showing a second example of the endoscope image pickup device of the embodiment of the present invention.

FIG. 8 is a schematic perspective view showing a connecting member of the second example of the endoscope image pickup device of the embodiment of the present invention.

FIG. 9 is a schematic side view showing the second example of the endoscope image pickup device of the embodiment of the present invention.

FIG. 10 is a schematic top view showing the second example of the endoscope image pickup device of the embodiment of the present invention.

FIG. 11 is a schematic enlarged side view showing a part of the second example of the endoscope image pickup device of the embodiment of the present invention.

FIG. 12 is a schematic perspective view showing another example of the connecting member of the second example of the endoscope image pickup device of the embodiment of the present invention.

FIG. 13 is a schematic side view showing a third example of the endoscope image pickup device of the embodiment of the present invention.

### DESCRIPTION OF THE PREFERRED EMBODIMENTS

Hereinafter, an endoscope image pickup device and an endoscope of the embodiment of the present invention will be described in detail on the basis of suitable embodiments shown in the accompanying drawings.

It should be noted that the drawings to be described below are examples illustrating the present invention, and the present invention is not limited to the drawings shown below.

In the following, “to” indicating a numerical range includes numerical values described on both sides thereof. For example, in a case in which ε is a numerical value ε_{α} to a numerical value ε_{β}, a range of ε is a range including the numerical value ε_{α} and the numerical value ε_{β} and is represented by ε_{α}<ε<ε_{β} in mathematical symbols.

In the following description, “specific angle”, “parallel”, “perpendicular”, “orthogonal”, and the like include an error range generally allowed in the relevant technical field.

### Endoscope System

An endoscope system irradiates an observation site such as an inside of a body of a subject to be examined which is an observation target, with illumination light (not shown), picks up an image of the observation site, generates a display image of the observation site on the basis of an image signal obtained by the image pickup, and displays the display image.

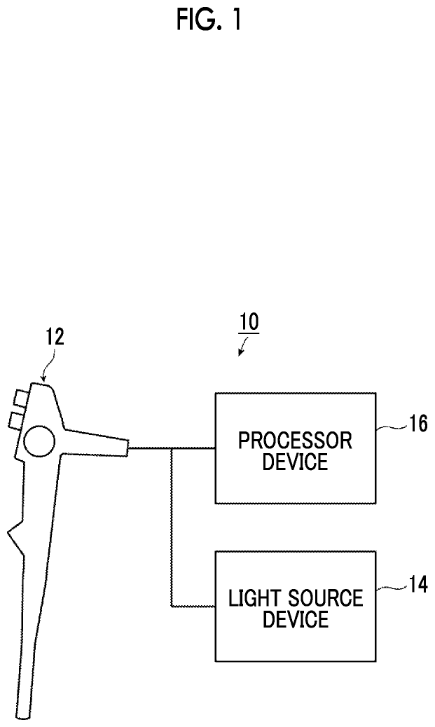

FIG. 1 is a schematic diagram showing an example of the endoscope system of the embodiment of the present invention.

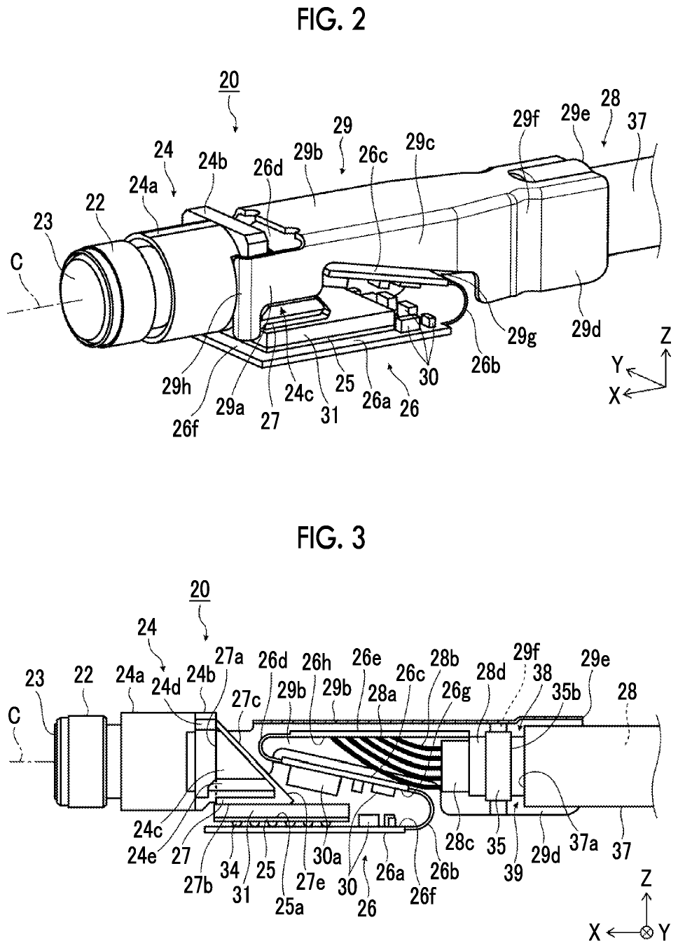

An endoscope system 10 has an endoscope 12, a light source device 14, and a processor device 16. The endoscope system 10 has the same configuration as a general endoscope except for a portion of an endoscope image pickup device 20 (see FIG. 2) of the endoscope 12, which will be described later.

The endoscope 12 has an endoscope image pickup device. In addition, although not shown in detail, the endoscope 12 has an insertion part to be inserted into a subject, an operation part connected to the insertion part, and a universal cord extending from the operation part, and the insertion part is composed of a distal end portion, a bendable portion connected to the distal end portion, and a soft portion that interconnects the bendable portion and the operation part. The endoscope image pickup device will be described later.

The distal end portion of the endoscope 12 is provided with an illumination optical system that emits illumination light for illuminating the observation site, or the endoscope image pickup device 20 (see FIG. 2) having an image pickup element, an image pickup optical system, or the like that picks up an image of the observation site. The bendable portion is bendable in a direction orthogonal to a longitudinal axis of the insertion part, and a bending operation of the bendable portion is operated at the operation part. In addition, the soft portion is relatively flexible so as to be deformable in conformity with a shape of an insertion path of the insertion part.

The operation part is provided with a button for operating an image pickup operation of the endoscope image pickup device 20 (see FIG. 2) of the distal end portion, a knob for operating the bending operation of the bendable portion, and the like. In addition, the operation part is provided with an introduction port into which a treatment tool, such as an electric scalpel, is introduced, and a treatment tool channel which reaches the distal end portion from the introduction port and through which the treatment tool, such as a forceps, is inserted is provided inside the insertion part.

A connector is provided at a terminal of the universal cord, and the endoscope 12 is connected to the light source device 14 that generates illumination light which is emitted from the illumination optical system of the distal end portion and to the processor device 16 that processes a video signal acquired by the endoscope image pickup device 20 (see FIG. 2) of the distal end portion, via the connector.

The processor device 16 processes the input video signal to generate video data of the observation site, and displays the generated video data on a monitor (not shown) or records the generated video data on a storage medium, such as a hard disk. The processor device 16 may be composed of a processor, such as a personal computer.

The light source device 14 is a light source device for illuminating an observation target site inside a body cavity by generating illumination light, such as white light consisting of three primary color lights, such as red light (R), green light (G), and blue light (B), or specific wavelength light, to supply the generated light to the endoscope 12, by propagating the supplied light through a light guide or the like provided inside the endoscope 12, and by emitting the propagated light from the illumination optical system of the distal end portion of the insertion part of the endoscope 12, in order to acquire the image signal of the observation target by picking up an image of the observation target site inside the body cavity through the endoscope image pickup device 20 (see FIG. 2) of the endoscope 12.

A light guide or an electrical wire group (signal cable) is accommodated inside the insertion part, the operation part, and the universal cord. The illumination light generated by the light source device 14 is guided to the illumination optical system of the distal end portion via the light guide, and at least one of signals or power is transmitted between the endoscope image pickup device 20 (see FIG. 2) of the distal end portion and the processor device 16 via the electrical wire group.

In addition, the endoscope system 10 may further comprise a water supply tank that stores wash water and the like, a suction pump that sucks a suction substance (also including supplied wash water and the like) in the body cavity, and the like. Further, the endoscope system 10 may comprise a supply pump that supplies wash water in the water supply tank or gas, such as external air, to a pipe line (not shown) in the endoscope, and the like.

### First Example of Endoscope Image Pickup Device

FIG. 2 is a schematic perspective view showing a first example of the endoscope image pickup device of the embodiment of the present invention, and FIG. 3 is a schematic side view showing the first example of the endoscope image pickup device of the embodiment of the present invention. A part of a connecting member 29 is omitted from the drawing of FIG. 3.

The endoscope image pickup device 20 shown in FIG. 2 acquires the image of the observation target. The endoscope image pickup device 20 has, for example, an image pickup lens 23, a lens barrel 22 that holds the image pickup lens 23, a holder 24, an image pickup element 25, a circuit board 26, a prism 27, and a signal cable 28. In addition, the endoscope image pickup device 20 has the connecting member 29. The holder 24 and the connecting member 29 are engaged with each other at an engaging portion 76 (see FIG. 5). An adhesive layer 74 (see FIG. 5) containing a filler is provided in the engaging portion 76 as described later.

Further, for example, an outer covering 28d (see FIG. 3) of the signal cable 28 is covered with a protective tube 37.

Here, a direction parallel to an optical axis C of the image pickup lens 23 is denoted by an X direction. Among two directions orthogonal to the optical axis C, one is denoted by a Y direction, and the rest is denoted by a Z direction. The Y direction corresponds to a width direction of the endoscope image pickup device 20, and the Z direction corresponds to a height direction of the endoscope image pickup device 20.

The image pickup lens 23 is an optical element that forms an image of light incident on the image pickup lens 23 on a light-receiving surface 25a of the image pickup element 25. The image pickup lens 23 is held by the lens barrel 22.

The lens barrel 22 is a tubular member and holds one or more image pickup lenses 23 therein. The lens barrel 22 holds the image pickup lens 23 such that the optical axis C of the image pickup lens 23 is perpendicular to an incident surface 27a (see FIG. 3) of the prism 27. The endoscope image pickup device 20 has, for example, three image pickup lenses 23, and the three image pickup lenses 23 are held by the lens barrel 22.

The configurations of the image pickup lens 23 and of the lens barrel 22 are not particularly limited. For example, a configuration may be employed in which one image pickup lens 23 is provided, or a configuration may be employed in which two or four or more image pickup lenses 23 are provided. Further, each image pickup lens 23 may be a convex lens or a concave lens.

The image pickup element 25 is an image pickup element that performs image pickup by converting light of which the image is formed by the image pickup lens 23 into an electrical signal through photoelectric conversion, and has the light-receiving surface 25a. In the image pickup element 25, the light of which the image is formed by the image pickup lens 23, that is, image pickup light of the observation target, is incident on the light-receiving surface 25a, and the image pickup light is photoelectrically converted, whereby image pickup is performed. The image pickup element 25 is a conventionally known image pickup element, and a Charge Coupled Device (CCD) type image sensor or a Complementary Metal Oxide Semiconductor (CMOS) image sensor can be used.

The image pickup element 25 is disposed on a side opposite to the lens barrel 22 with respect to the holder 24. As shown in FIGS. 2 and 3, for example, the image pickup element 25 is electrically connected to a front surface 26f of a first plane portion 26a of the circuit board 26 via a bump 34 having conductivity. Further, as shown in FIG. 3, the image pickup element 25 is mounted on the circuit board 26 such that the light-receiving surface 25a is parallel to the optical axis C of the image pickup lens 23. The mounting means electrical connection.

An underfill layer (not shown) can also be provided between the image pickup element 25 and the circuit board 26 in order to firmly connect the image pickup element 25 and the circuit board 26 to each other.

The bump 34 is formed of a metal or an alloy. In more detail, the bump 34 is formed of solder. The bump 34 formed of solder is also referred to as a solder ball. The bump 34 is not limited to solder or the like as long as the image pickup element 25 and the circuit board 26 can be electrically connected to each other. In addition, the image pickup element 25 and the circuit board 26 may be directly electrically connected to each other.

Further, a stress is generated in a joint portion between the image pickup element 25 and the circuit board 26, for example, in the bump 34 because of a difference in thermal expansion coefficient between the image pickup element 25 and the circuit board 26, but the underfill layer relaxes this stress. With the underfill layer, the image pickup element 25 and the circuit board 26 are firmly connected to each other, and the reliability of the electrical connection is increased, so that the endoscope image pickup device 20 having high reliability can be obtained.

An underfill agent that constitutes the underfill layer is not particularly limited, and an underfill agent that is used as a sealing resin between the image pickup element 25 and the circuit board 26 can be appropriately used. For example, as the underfill agent, a one-pack heat-curable type epoxy resin is used. In this case, the underfill agent is supplied, and then is heated and held at a predetermined temperature, whereby the underfill layer is formed.

The circuit board 26 is a board on which the image pickup element 25 is mounted. In addition, for example, electronic components 30 and 30a are mounted on the circuit board 26, in addition to the image pickup element 25. The electronic components 30 and 30a are for driving the image pickup element 25 and are not particularly limited, and examples thereof include a voltage regulator, a resistor, and a capacitor. The voltage regulator is a device that stabilizes a voltage to the image pickup element 25, and outputs a constant voltage to the image pickup element 25.

In the example shown in the drawing, the circuit board 26 has the first plane portion 26a, a second plane portion 26c connected by the first plane portion 26a and a first bent portion 26b, and a third plane portion 26e connected by the second plane portion 26c and a second bent portion 26d. The first plane portion 26a and the third plane portion 26e are parallel to the optical axis C of the image pickup lens 23. The second plane portion 26c is inclined at an angle with respect to the optical axis C of the image pickup lens 23. That is, the second plane portion 26c is not parallel to the optical axis C. The second plane portion 26c is inclined such that the second bent portion 26d is located on an upper side in the Z direction with respect to the first bent portion 26b.

Further, the circuit board 26 has a plurality of connection terminals (not shown) through which signals or power is input and output to and from the image pickup element 25 and the electronic components 30 and 30a and which are provided on a back surface 26h (see FIG. 3) of the third plane portion 26e (see FIG. 3) facing the second plane portion 26c (see FIG. 3). Signal lines 28a (see FIG. 3) of the signal cable 28 are electrically connected to the connection terminals.

The circuit board 26 is formed of, for example, a flexible board, and is formed of, for example, a flexible printed board.

The image pickup element 25 is mounted on the front surface 26f of the first plane portion 26a as shown in FIGS. 2 and 3. In addition, the electronic component 30 is also mounted on the front surface 26f of the first plane portion 26a.

The electronic components 30 and 30a are mounted on a back surface 26g of the second plane portion 26c facing the front surface 26f of the first plane portion 26a. Since the second plane portion 26c is inclined with respect to the first plane portion 26a, a region having a wide space is formed between the first plane portion 26a and the second plane portion 26c. Therefore, the electronic component 30a having a large size can be mounted. For example, in the back surface 26g of the second plane portion 26c, a height of the electronic component 30a mounted on a second bent portion 26d side is higher than a height of the electronic component 30 mounted on a first bent portion 26b side. In this way, electronic components having various sizes can be mounted, and the space of the endoscope image pickup device 20 can be effectively utilized.

As described above, the connection terminals (not shown) are provided on the back surface 26h of the third plane portion 26e facing the second plane portion 26c. The dispositions of the image pickup element 25, the electronic components 30 and 30a, the connection terminals, and the like on the circuit board 26 are not particularly limited.

The signal lines 28a (see FIG. 3) of the signal cable 28 are electrically connected to the connection terminals provided in the back surface 26h (see FIG. 3) of the third plane portion 26e of the circuit board 26, and the image pickup element 25 and the signal cable 28 are electrically connected to each other. Light is converted into an electrical signal by the image pickup element 25, and this electrical signal is transmitted via the signal cable 28. The signal cable 28 is inserted through the insertion part, the operation part, the universal cord, or the like of the endoscope and is electrically connected to the processor device 16 (see FIG. 1).

The signal cable 28 is not particularly limited as long as the signal cable 28 has the signal lines 28a and the outer covering 28d forming an outer periphery thereof. For example, as shown in FIG. 3, the signal cable 28 has the plurality of signal lines 28a, a covering layer 28b that covers each signal line 28a, a shielded conductor 28c provided around all of the plurality of signal lines 28a covered with the covering layers 28b, and the outer covering 28d that covers the shielded conductor 28c. The signal cable 28 is a multi-core cable into which the plurality of signal lines 28a are bundled, around which the shielded conductor 28c is provided, and which is accommodated inside the cylindrical outer covering 28d.

As described above, the outer covering 28d constitutes the outer periphery of the signal cable 28. The covering layer 28b, the shielded conductor 28c, and the outer covering 28d each have, for example, a cylindrical shape. Further, the shielded conductor 28c of the signal cable 28 is referred to as a shield. The signal cable 28 has, for example, five signal lines 28a. The number of the signal lines 28a depends on the configuration of the endoscope image pickup device 20, is not particularly limited, and may be two, three, four, or six or more.

The prism 27 is disposed between the lens barrel 22 and the image pickup element 25 via a cover glass 31. The prism 27 guides light that has passed through the image pickup lens 23 to the light-receiving surface 25a of the image pickup element 25. The prism 27 is, for example, a right-angle prism in which an incident surface 27a and an emission surface 27b are orthogonal to each other. Further, the prism 27 has an inclined surface 27c interconnecting the incident surface 27a and the emission surface 27b. The inclined surface 27c is a reflecting surface 27e of the prism 27.

The prism 27 bends light that has passed through the image pickup lens 23 held by the lens barrel 22 in the inclined surface 27c, that is, in the reflecting surface 27e, by, for example, 90° to change an optical path, and guides the light to the light-receiving surface 25a of the image pickup element 25. The light transmitted through the image pickup lens 23 is incident on the prism 27, is reflected in the inclined surface 27c of the prism 27, that is, in the reflecting surface 27e, and is incident on the light-receiving surface 25a of the image pickup element 25. The light transmitted through the image pickup lens 23 is light including information on the observation target.

For example, the prism 27 is disposed such that the incident surface 27a faces a surface on a proximal end side of the lens barrel 22. Further, the prism 27 is disposed such that the emission surface 27b faces the light-receiving surface 25a of the image pickup element 25. In this case, the prism 27 is disposed on the cover glass 31 such that the emission surface 27b faces the cover glass 31.

The cover glass 31 is disposed on the light-receiving surface 25a of the image pickup element 25 and protects the light-receiving surface 25a. The prism 27 and the cover glass 31 are bonded to each other with, for example, a photocurable adhesive. A configuration may be employed in which no cover glass 31 is provided.

The holder 24 is a member that holds the lens barrel 22 and the prism 27. The holder 24 is a substantially tubular member, and the lens barrel 22 is fitted to an inside of the tubular portion, whereby the holder 24 holds the lens barrel 22. An inner surface of the holder 24 and an outer peripheral surface of the lens barrel 22 are bonded and fixed.

As an adhesive for bonding the holder 24 and the lens barrel 22 to each other, various known adhesives used in a conventional endoscope can be used. This point is also the same for the adhesive for bonding other members to each other.

The holder 24 has a polygonal flange portion 24b provided on an edge surface on the proximal end side of an attachment tubular portion 24a. A restricting member 24c is provided at each of both ends of the flange portion 24b in the Y direction. A front surface of the restricting member 24c is a side surface 24m of the holder 24 (see FIG. 5).

The prism 27 is disposed between the restricting members 24c, and the incident surface 27a is in contact with the flange portion 24b in a state of being sandwiched between the restricting members 24c. With this, the prism 27 is positioned in the X direction. The holder 24 holds the lens barrel 22 and the prism 27 at predetermined positions so that positions of the lens barrel 22 and of the prism 27 relative to each other, that is, positions of the lens barrel 22 and of the light-receiving surface 25a of the image pickup element 25 relative to each other are fixed. The emission surface 27b of the prism 27 and the image pickup element 25 face each other.

For example, a projecting portion 24d and a projecting portion 24e are disposed on the side surface 24m (see FIG. 5) of the holder 24 so as to face each other at an interval along the height direction orthogonal to the optical axis C of the image pickup lens 23. An arm portion 29a of the connecting member 29 is provided on the side surface 24m between the projecting portion 24d and the projecting portion 24e. The above-described height direction is the Z direction as described above. The holder 24 and the connecting member 29 will be described in detail later.

Here, the position of the lens barrel 22 relative to the holder 24 in a direction of the optical axis C of the image pickup lens 23 is adjusted so as to be in focus on the light-receiving surface 25a of the image pickup element 25 so that the lens barrel 22 is bonded and fixed to the holder 24. The direction of the optical axis C is an extending direction of the optical axis C of the image pickup lens 23. The direction of the optical axis C of the image pickup lens 23 is a direction parallel to the X direction.

The connecting member 29 connects the holder 24 and the signal cable 28 to each other. The connecting member 29 holds the signal cable 28 therein. For example, as shown in FIG. 2, the connecting member 29 is a member formed by bending one plate material. Specifically, the connecting member 29 has a shape in which one plate material extends in the direction of the optical axis C and is bent at two bending portions. Therefore, the connecting member 29 has a substantially C shape in a cross-section perpendicular to the direction of the optical axis C. The connecting member 29 is disposed so as to include the connection terminals (a connection part with the signal cable 28) provided on the circuit board 26 on an inner side of the substantially C shape. That is, as shown in FIG. 2, the connecting member 29 has a base portion 29b that covers the circuit board 26 from the upper side in the drawing, and side surface portions 29c that are each formed by being bent at an end of the base portion 29b. The side surface portion 29c has, for example, an edge 29g parallel to the second plane portion 26c of the circuit board 26. The edge 29g is located on a base portion 29b side with respect to the second plane portion 26c, and the second plane portion 26c of the circuit board 26 is exposed in a case in which the circuit board 26 is covered with the connecting member 29 from above.

The connecting member 29 covers a part of the third plane portion 26e or the like of the circuit board 26, the prism 27, and a distal end portion of the signal cable 28, and also serves as a cover member for the circuit board 26, the prism 27, and the signal cable 28. Further, the connecting member 29 also functions as a protective member for the circuit board 26, the prism 27, and the signal cable 28.

The connecting member 29 has a pair of the arm portions 29a facing each other and provided on a distal end side. The distal end portions 29h of the pair of arm portions 29a are each bent inward to form a claw portion. That is, the distal end portions 29h are each bent in parallel with the Y direction. The pair of arm portions 29a sandwich the flange portion 24b of the holder 24, and the distal end portions 29h of the arm portions 29a are engaged so that the connecting member 29 is fixed to the holder 24.

In addition, the connecting member 29 has a holding portion 29d that is provided on a proximal end side of the side surface portion 29c and that holds the signal cable 28. The holding portion 29d is bent along the outer covering 28d, and a width in the Y direction becomes narrower from the arm portion 29a toward the holding portion 29d. The signal cable 28 is fixed and held inside the holding portion 29d. The holding portion 29d is a member connected to the side surface portion 29c.

In addition, the connecting member 29 is connected to the holder 24 and to the signal cable 28 so that the connection between the connection terminals and the signal lines 28a is prevented from being disconnected because the connection part between the connection terminals provided on the circuit board 26 and the signal lines 28a of the signal cable 28 is pulled, for example, in a case in which the signal cable 28 is pulled.

A metal material for forming the connecting member 29 is not particularly limited, but a metal material having a high thermal conductivity is preferable. In consideration of workability, availability, strength, and the like, the connecting member 29 is preferably a stainless steel or a copper alloy.

The protective tube 37 protects the signal cable 28, prevents the signal cable 28 from breaking, and prevents the signal lines 28a from being disconnected.

As shown in FIG. 3, the protective tube 37 is provided such that a distal end 37a is located on a lens barrel 22 side with respect to a rear edge surface 29e of the connecting member 29 provided on a side opposite to the lens barrel 22, and on a rear edge surface 29e side with respect to a fixing member 35. The protective tube 37 is provided on the signal cable 28 so as to overlap the connecting member 29. The distal end 37a of the protective tube 37 is an end of the protective tube 37 provided on the lens barrel 22 side.

The arm portion 29a of the connecting member 29 and the flange portion 24b of the holder 24, and the holding portion 29d of the connecting member 29 and the outer covering 28d of the signal cable 28 are each bonded and fixed to each other by using, for example, an adhesive 38. In a case of being bonded and fixed to each other, the adhesive 38 is in a cured state. The present invention is not limited to the bonding and fixing using the adhesive 38 as long as the holding portion 29d of the connecting member 29 and the outer covering 28d of the signal cable 28 can be fixed.

Hereinafter, the holder 24 and the connecting member 29 will be described.

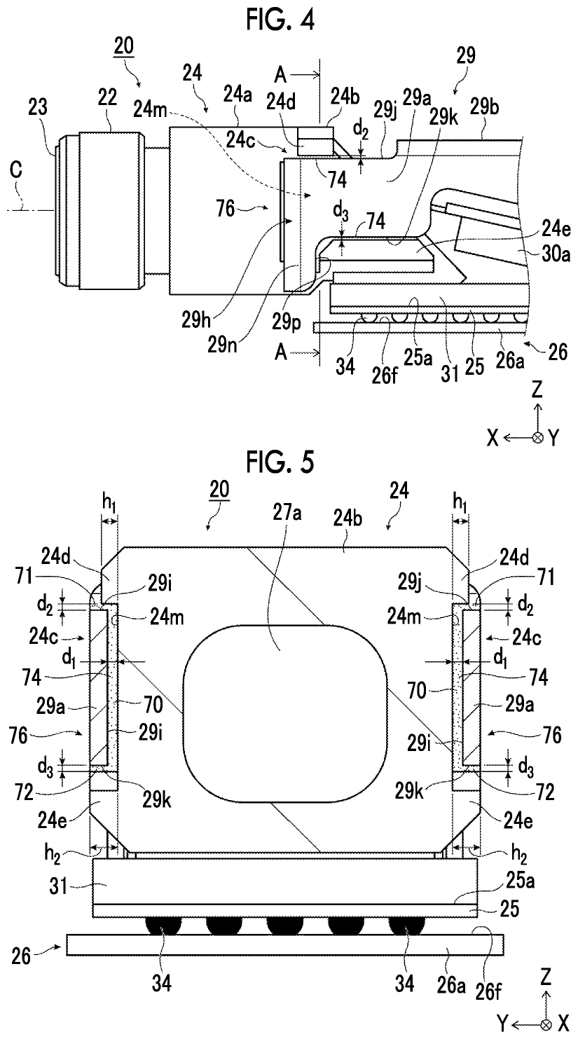

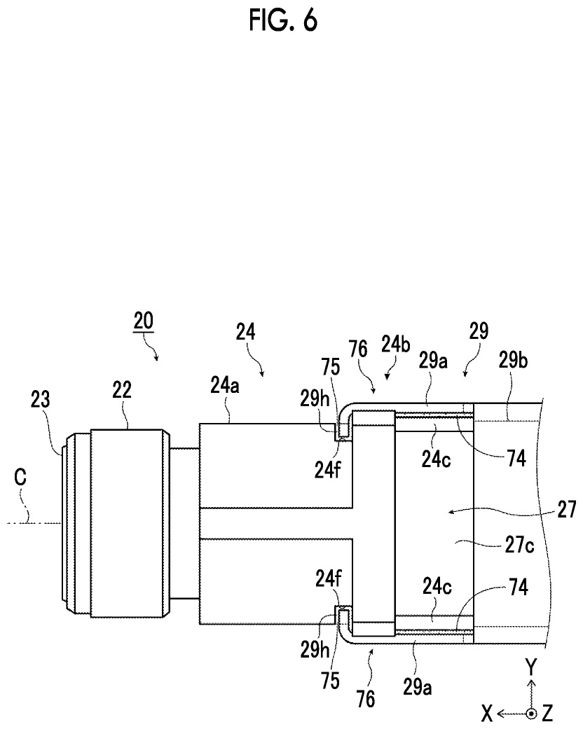

FIG. 4 is a schematic enlarged side view showing a part of the first example of the endoscope image pickup device of the embodiment of the present invention. FIG. 5 is a schematic cross-sectional view taken along line A-A of the first example of the endoscope image pickup device of the embodiment of the present invention. FIG. 6 is a schematic enlarged plan view showing a part of the first example of the endoscope image pickup device of the embodiment of the present invention. In FIGS. 4 to 6, the same constituents as those of the endoscope image pickup device 20 shown in FIGS. 2 and 3 are designated by the same reference numerals, and detailed descriptions thereof will be omitted.

As described above, the arm portion 29a of the connecting member 29 is provided on the side surface 24m between the projecting portion 24d and the projecting portion 24e. As shown in FIG. 5, there is a first gap 70 between the side surface 24m and a back surface 29i of the arm portion 29a of the connecting member 29. The adhesive layer 74 containing a filler is provided in the first gap 70, and the arm portion 29a of the connecting member 29 is bonded to the side surface 24m of the holder 24 via the adhesive layer 74 containing a filler. The adhesive layer 74 containing a filler is also simply referred to as the adhesive layer 74.

A part where the arm portion 29a and the holder 24 are bonded to each other via the adhesive layer 74 is the engaging portion 76 (see FIG. 5) between the holder 24 and the connecting member 29. The adhesive layer 74 is provided in the engaging portion 76. The adhesive layer 74 containing a filler is used, so that it is possible to realize a firm fixation and a stable connection strength in the engaging portion 76. As a result, the strength of the endoscope image pickup device 20 can be increased, and the strength can be stably maintained.

For example, the adhesive layer 74 is formed by fitting the arm portion 29a to the side surface 24m (see FIG. 5) of the holder 24 by spreading the pair of arm portions 29a wider than the projecting portions 24d after filling a space between the projecting portion 24d and the projecting portion 24e of the holder 24 with a filler-containing adhesive, and then by curing the filler-containing adhesive.

A thickness of the adhesive layer 74 is preferably 5 μm or more from the viewpoint of securing the thickness of the adhesive layer 74 and reducing variation in the bonding strength. For this reason, a width d₁ of the first gap 70 in FIG. 5 is preferably 5 μm or more. For example, the width d₁ of the first gap 70 is preferably wider than an average particle diameter of the filler contained in the adhesive layer 74 in order to leave the filler of the filler-containing adhesive and maintain the thickness of the adhesive layer 74. The filler-containing adhesive is also simply referred to as an adhesive.

An upper limit of the thickness of the adhesive layer 74 is about 100 to 120 μm. The thickness of the adhesive layer 74 is more preferably 30 to 70 μm from the fact that the variation in the bonding strength can be further reduced. An upper limit of the width d₁ of the first gap 70 in FIG. 5 is about 100 to 120 μm and more preferably 30 to 70 μm, as in the adhesive layer 74, from the viewpoint of securing the thickness of the adhesive layer 74.

The projecting portion 24d protrudes in a direction perpendicular to the side surface 24m with respect to the arm portion 29a in a state in which the arm portion 29a is bonded to the side surface 24m (see FIG. 5) of the holder 24 via the adhesive layer 74. The projecting portion 24d protrudes in the Y direction in FIGS. 4 and 5. That is, the projecting portion 24d protrudes outward of the connecting member 29 with respect to the arm portion 29a. Therefore, in a case in which the arm portion 29a is attached to the restricting member 24c of the holder 24, it is necessary to fit the arm portion 29a to the side surface 24m by spreading the arm portion 29a wider than the projecting portion 24e. The projecting portion 24d having the above-described configuration is provided, so that the adhesive is prevented from being removed by the arm portion 29a in a case in which the arm portion 29a is bonded to the side surface 24m (see FIG. 5) of the holder 24 by using the filler-containing adhesive.

It is preferable that a height h₁ of the projecting portion 24d provided on the side surface of the holder 24 is larger than the average particle diameter of the filler. With this, in a case in which the filler-containing adhesive is provided in the first gap 70, the filler contained in the adhesive remains, and the adhesive layer 74 can be reliably provided.

The height h₁ of the projecting portion 24d provided on the side surface of the holder 24 is a distance from the side surface 24m to the highest point of the projecting portion 24d.

A height h₂ of the projecting portion 24e provided on the side surface of the holder 24 is a distance from the side surface 24m to the highest point of the projecting portion 24e.

It is preferable that the height h₂ of the projecting portion 24e is also larger than the average particle diameter of the filler of the adhesive layer 74. With this, the filler contained in the adhesive remains, and the adhesive layer 74 can be reliably provided.

A cross-sectional image of the endoscope image pickup device 20 as shown in FIG. 5 is acquired for each of the thickness of the adhesive layer 74, the height h₁ of the projecting portion 24d, and the height h₂ of the projecting portion 24e. The acquired cross-sectional image is used to specify a corresponding part corresponding to each of the thickness of the adhesive layer 74, the height h₁ of the projecting portion 24d, and the height h₂ of the projecting portion 24e. A length is measured using a ruler by, for example, enlarging an image of the corresponding part. The measured values are the thickness of the adhesive layer 74, the height h₁ of the projecting portion 24d, and the height h₂ of the projecting portion 24e.

The above-described measurement of the thickness of the adhesive layer 74, the height h₁ of the projecting portion 24d, and the height h₂ of the projecting portion 24e also include entering the acquired cross-sectional image of the endoscope image pickup device 20 into a computer and using software to measure the length of the corresponding part corresponding to each of the thickness of the adhesive layer 74, the height h₁ of the projecting portion 24d, and the height h₂ of the projecting portion 24e described above.

As shown in FIGS. 4 and 5, there is a second gap 71 between the projecting portion 24d and an upper edge 29j of the arm portion 29a. The adhesive layer 74 is provided in the second gap 71. There is a third gap 72 between the projecting portion 24e and a lower edge 29k of the arm portion 29a. The adhesive layer 74 is provided in the third gap 72. A width d₂ of the second gap 71 and a width d₃ of the third gap 72 are gaps between the projecting portions 24d and 24e facing each other in the height direction, and the arm portion 29a.

It is preferable that the width d₂ of the second gap 71 and the width d₃ of the third gap 72 are each wider than the average particle diameter of the filler contained in the adhesive layer 74. With this, in a case in which the arm portion 29a of the connecting member 29 is provided on the side surface 24m between the projecting portion 24d and the projecting portion 24e, the arm portion 29a of the connecting member 29 can be reliably fitted to the side surface 24m even in a case in which there is the filler-containing adhesive.

Further, the width d₂ of the second gap 71 and the width d₃ of the third gap 72 are each preferably 5 μm or more as in the first gap 70 in FIG. 5 described above, and with this, for example, the thickness of the adhesive layer 74 can be maintained while leaving the filler of the filler-containing adhesive. Further, the adhesive layer 74 can secure a sufficient thickness for bonding and has a sufficient bonding strength. An upper limit of each of the width d₂ of the second gap 71 and the width d₃ of the third gap 72 is about 100 to 120 μm and more preferably 30 to 70 μm, as in the first gap 70 of FIG. 5 described above, from the viewpoint of securing the thickness of the adhesive layer 74. In this case, the upper limit of the thickness of the adhesive layer 74 is about 100 to 120 μm and more preferably 30 to 70 μm as described above from the fact that the variation in the bonding strength can be further reduced.

In the measurement of each of the width d₁ of the first gap 70, the width d₂ of the second gap 71, and the width d₃ of the third gap 72, an image from an arm portion 29a side of the endoscope image pickup device 20 is acquired. The acquired image is used to specify a corresponding part corresponding to each of the width d₁ of the first gap 70, the width d₂ of the second gap 71, and the width d₃ of the third gap 72. A length is measured using a ruler by, for example, enlarging an image of the corresponding part. The measured values are the width d₁ of the first gap 70, the width d₂ of the second gap 71, and the width d₃ of the third gap 72.

The above-described measurement of each of the width d₁ of the first gap 70, the width d₂ of the second gap 71, and the width d₃ of the third gap 72 also include entering the acquired image of the endoscope image pickup device 20 into the computer and using software to measure the length of the corresponding part corresponding to each of the width d₁ of the first gap 70, the width d₂ of the second gap 71, and the width d₃ of the third gap 72 described above.

As shown in FIG. 4, the arm portion 29a has a latch portion 29n that latches onto the projecting portion 24e. The latch portion 29n is hooked on the projecting portion 24e, the position of the connecting member 29 in the optical axis direction of the optical axis C of the image pickup lens 23 is restricted, and the connecting member 29 does not easily come off. With this, a tensile strength of the connecting member 29 in the optical axis direction is increased.

The latch portion 29n of the arm portion 29a is provided, for example, on the lens barrel 22 side with respect to the distal end of the projecting portion 24e and extends to an image pickup element 25 side. The latch portion 29n has, for example, an L-shaped latch shape. In the latch portion 29n, an inner edge 29p is hooked on the projecting portion 24e to restrict the movement of the connecting member 29. The inner edge 29p is an edge of the latch portion 29n on a signal cable 28 (see FIG. 3) side.

A shape of the latch portion 29n is not particularly limited to the L-shaped latch shape shown in FIG. 4 as long as the latch portion 29n is hooked on the projecting portion 24e.

As described above, the distal end portions 29h of the pair of arm portions 29a are bent inward with each other. The distal end portion 29h of the arm portion 29a forms a claw. As shown in FIG. 6, the holder 24 has a groove 24f that is engaged with the distal end portion 29h of the arm portion 29a. The groove 24f is formed linearly along the Z direction. A length of the groove 24f is preferably longer than a length of the distal end portion 29h of the arm portion 29a in the Z direction in order to reliably perform engagement with the arm portion 29a. In addition, a depth of the groove 24f is preferably deeper than a length of the distal end portion 29h in order to reliably enter the distal end portion 29h of the arm portion 29a.

The pair of arm portions 29a sandwich the flange portion 24b of the holder 24, and the distal end portion 29h of the arm portion 29a enters the groove 24f and is engaged and locked. In a state in which the distal end portion 29h is locked to the groove 24f, the groove 24f is filled with the filler-containing adhesive and the filler-containing adhesive is cured, whereby an adhesive layer 75 containing a filler is provided. The distal end portion 29h of the arm portion 29a is in the adhesive layer 75. The distal end portion 29h of the arm portion 29a is bonded in a state of being hooked in the groove 24f so that the position of the connecting member 29 in the optical axis direction of the optical axis C of the image pickup lens 23 is restricted and the tensile strength of the connecting member 29 in the optical axis direction is increased.

The adhesive layer 75 has the same configuration as that of the adhesive layer 74 and has a filler and a resin containing a filler. For example, the adhesive layer 75 forms an adhesive layer by filling the groove 24f with the filler-containing adhesive and curing the filler-containing adhesive in a state in which the distal end portion 29h of the arm portion 29a is engaged with the groove 24f.

In the endoscope image pickup device 20, as shown in FIG. 3, the fixing member 35 that is compressed to fix the signal cable 28 from above the outer covering 28d of the signal cable 28 toward the center may be provided on a distal end of the signal cable 28 which is an end on a connecting member 29 side.

The fixing member 35 is provided on the outer covering 28d of the signal cable 28, and is tightened to fix the outer covering 28d to the signal lines 28a of the signal cable 28. The fixing member 35 is, for example, an annular member, and the fixing member 35 is tightened by being compressed toward the center of the signal cable 28, that is, by being squeezed, to fix the outer covering 28d to the signal lines 28a of the signal cable 28.

The fixing member 35 is not limited to the annular shape as long as the outer covering 28d can be fixed to the signal lines 28a of the signal cable 28, and may be a polygonal annular member. Alternatively, the fixing member 35 may have a configuration in which a gap is provided without being continuous around the periphery, instead of the annular shape, or may have, for example, a C-ring shape. In this case, distant end parts are brought closer to each other by being squeezed such that the distant end parts are close to each other, whereby an opening portion of the fixing member 35 is reduced and the outer covering 28d is fixed to the signal lines 28a of the signal cable 28. Further, the fixing member 35 is formed of, for example, a metal. The metal forming the fixing member 35 also includes an alloy.

In addition, as described above, since the signal cable 28 used in the endoscope image pickup device 20 has a structure in which the plurality of signal lines 28a are bundled by the outer covering 28d and the signal lines 28a are easily damaged, protection is required by the outer covering 28d or the connecting member 29. Since the connecting member 29 is formed of a metal or the like and the holding portion 29d of the connecting member 29 has a rapid change in stiffness, a load is largely concentrated on the signal cable 28. For this reason, in a case in which the outer covering 28d is displaced and the signal line 28a is exposed to the outside of the connecting member 29 because of the bending operation of the endoscope or sliding with other contents, the signal line 28a is damaged in the vicinity of the holding portion 29d of the connecting member 29.

However, the fixing strength of the outer covering 28d of the signal cable 28 is increased by providing the fixing member 35 on the outer covering 28d of the signal cable 28, and then the protective tube 37 is fixed by overlapping the connecting member 29 as described above, so that a decrease in the bonding area of the outer covering 28d of the signal cable 28 is supplemented with the fixing strength of the fixing member 35 and the protective tube 37 improves the strength near the holding portion 29d of the connecting member 29. With this, the endoscope image pickup device 20 in which the joining strength of the signal cable 28 is high and the joining of the signal cable 28 is highly reliable can be obtained. As a result, disconnection of the signal cable 28 is also suppressed.

In a case of performing the boning and fixing by using the adhesive 38, it is preferable that the protective tube 37 is softer than the cured adhesive 38. The protective tube 37 is made softer than the cured adhesive 38, so that the protective tube 37 can be easily bent, and the load applied to the signal cable 28 at the holding portion 29d of the connecting member 29 is reduced. The protective tube 37 is formed of, for example, polytetrafluoroethylene (PTFE). In this case, for example, an epoxy resin-based adhesive is used as the adhesive 38. As the adhesive 38, for example, a silicone-based adhesive or an acrylic adhesive can be used in addition to the epoxy-based adhesive.

In addition, it is preferable to dispose the protective tube 37 by providing a gap 39 between an end 35b of the fixing member 35 provided on a holding portion 29d side of the connecting member 29 and the distal end 37a of the protective tube 37. The fixing member 35 and the protective tube 37 are provided by providing the above-described gap 39, so that it is possible to secure the bonding area of the adhesive 38. With the above-described gap 39, the signal cable 28 can be more reliably fixed to the connecting member 29. Further, in a case in which the signal cable 28 is pulled to the side opposite to the lens barrel 22, the fixing member 35 is caught by the adhesive 38, the signal cable 28 is prevented from being pulled out, and the damage to the signal cable 28 can be suppressed.

The gap 39 is preferably 0.5 mm or more because the bonding area of the adhesive 38 can be secured. Since the larger the bonding range is, the stronger the bonding strength is, an upper limit of the gap 39 is not particularly limited. However, from the structural restrictions of the endoscope image pickup device 20, the upper limit of the gap 39 is about 3 mm. The gap 39 is measured by using a micrometer or a caliper.

For example, the fixing member 35 and the protective tube 37 described above are assembled by using a holding device or equipment capable of positioning and assembling each component at a predetermined position.

In the endoscope image pickup device 20, an observational image captured by the image pickup element 25 from the image pickup lens 23 is formed on the light-receiving surface 25a of the image pickup element 25 and converted into an electrical signal, and this electrical signal is output to the processor device 16 (see FIG. 1) via the signal cable 28 and is converted into a video signal, and an observation image is displayed on the monitor connected to the processor device 16.

Further, for example, it is preferable that the connecting member 29 holds the signal cable 28 therein and has a recessed portion 29f protruding inward, and that at least a part of the recessed portion 29f overlaps the fixing member 35. With this configuration, in a case in which the connecting member 29 and the signal cable 28 are fixed to each other by using the adhesive 38, the connecting member 29 and the signal cable 28 can be fixed more firmly. Further, the recessed portion 29f makes it possible to easily position the center of the connecting member 29 and the center of the signal cable 28. The recessed portion 29f is formed, for example, by locally narrowing down the side surface portion 29c of the connecting member 29.

In the endoscope image pickup device 20, as shown in FIG. 3, the inclined surface 27c of the prism 27 faces the second bent portion 26d. It is preferable that at least a part of the second bent portion 26d of the circuit board 26 overlaps the inclined surface 27c of the prism 27 when viewed from a direction perpendicular to the light-receiving surface 25a of the image pickup element 25, that is, from the Z direction in FIG. 3. With this, the second bent portion 26d enters a space on an inclined surface 27c side of the prism 27, and the space on the inclined surface 27c side of the prism 27 is effectively used so that a length of the endoscope image pickup device 20 in the direction of the optical axis C can be shortened and the endoscope image pickup device 20 can be miniaturized in the direction of the optical axis C.

In the endoscope image pickup device 20, it is preferable that a part of the inclined surface 27c of the prism 27 and a part of the second bent portion 26d of the circuit board 26 are connected to each other by a photocurable adhesive (not shown), and that a part of the first bent portion 26b and/or a part of the second plane portion 26c, and a part of the signal cable 28 and/or the third plane portion 26e are connected to each other by a photocurable adhesive (not shown). With this, the shape of the circuit board 26 can be maintained, and the manufacturing time of the endoscope image pickup device 20 can be shortened.

The photocurable adhesive is, for example, an adhesive that is cured by ultraviolet light having a wavelength of about 100 to 400 nm, visible light having a wavelength of more than 400 and less than 780 nm, infrared light having a wavelength of about 780 nm to 1 mm, or the like. The photocurable adhesive is, for example, an epoxy resin-based photocurable adhesive, an acrylic resin-based photocurable adhesive, or a silicone-based photocurable adhesive. In addition, an adhesive in which photo-curing and heat-curing are used in combination may be used. This photocurable adhesive can also be used for the above-described bonding between the prism 27 and the cover glass 31.

Both the first bent portion 26b and the second bent portion 26d are each formed of a curved surface. Curvature radii of the first bent portion 26b and the second bent portion 26d may be the same as or different from each other. As shown in FIG. 4, the first bent portion 26b has a larger curvature radius than the second bent portion 26d. The curvature radii of the first bent portion 26b and of the second bent portion 26d are adjusted so that a space between the first plane portion 26a and the second plane portion 26c, and a space between the second plane portion 26c and the third plane portion 26e can be adjusted.

The first bent portion 26b and the second bent portion 26d are not limited to being configured only by a curved surface as long as a curved surface is provided, and for example, a configuration may be employed in which the first bent portion 26b and the second bent portion 26d each have a plane and a curved surface.

The curvature radius is obtained as follows. First, an image of the circuit board 26 from a side surface direction is acquired. The acquired image is used to specify a corresponding part corresponding to each of the curvature radii of the first bent portion 26b and of the second bent portion 26d. A curve is fitted to the corresponding part, and the curvature radius of the curve is measured by using a ruler. The measured value is the curvature radius.

The above-described measurement of the curvature radius also includes entering the acquired image of the circuit board 26 into the computer and using software to measure the curvature radii of the first bent portion 26b and the second bent portion 26d. Fitting the curve to the corresponding part corresponding to the curvature radius and measuring the curvature radius of the curve by using a ruler also includes performing the measurement by using software on the computer.

### Second Example of Endoscope Image Pickup Device

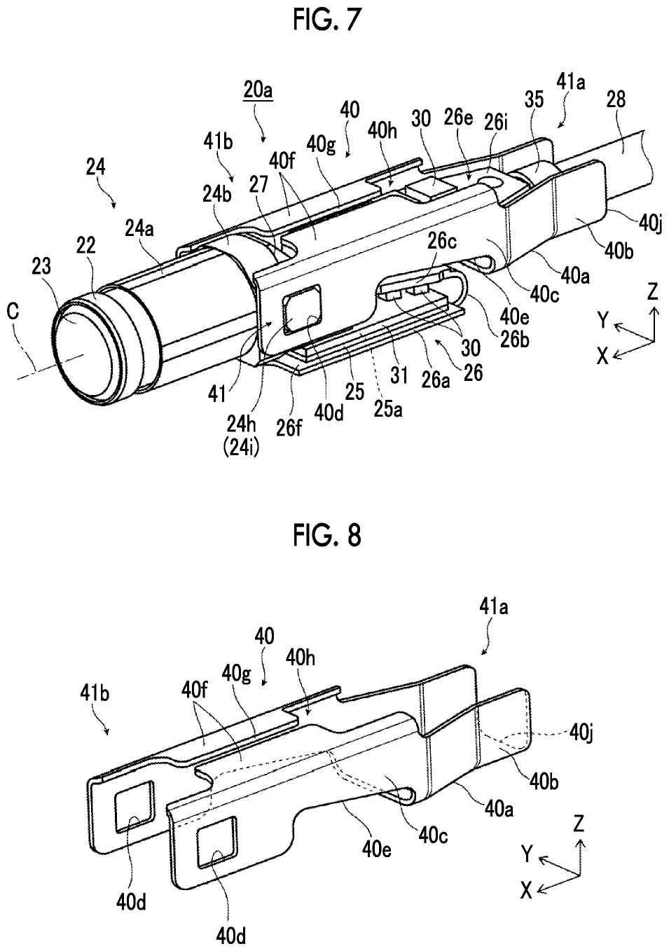

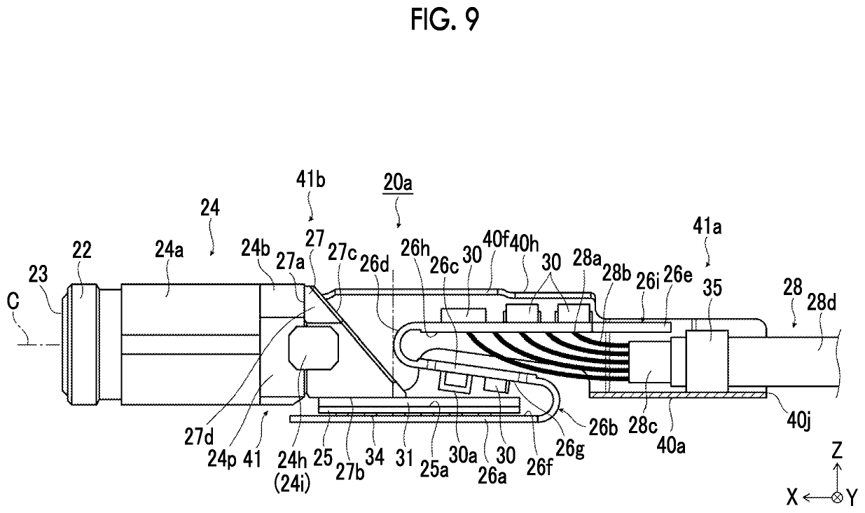

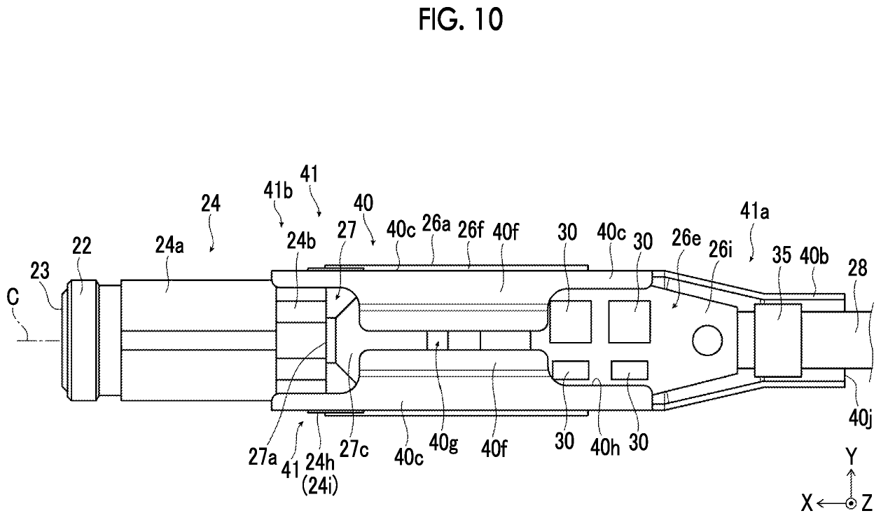

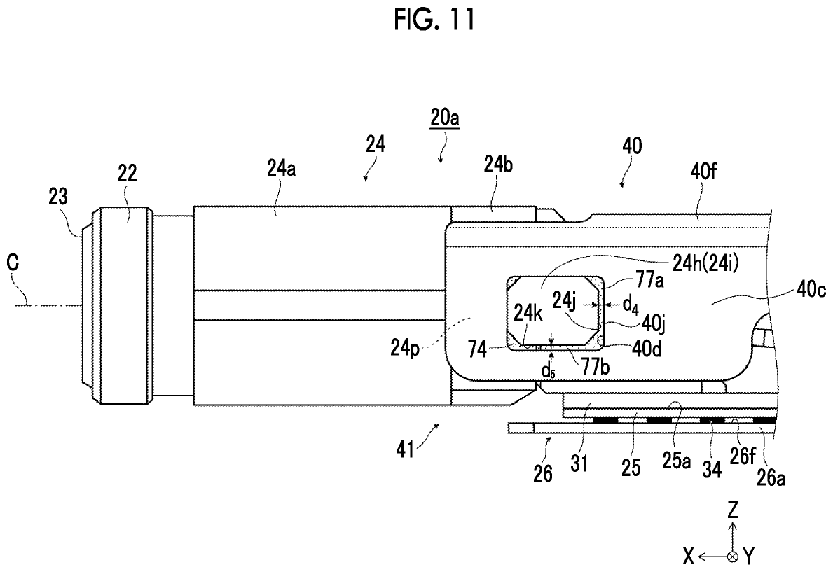

FIG. 7 is a schematic perspective view showing a second example of the endoscope image pickup device of the embodiment of the present invention, and FIG. 8 is a schematic perspective view showing a connecting member of the second example of the endoscope image pickup device of the embodiment of the present invention. FIG. 9 is a schematic side view showing the second example of the endoscope image pickup device of the embodiment of the present invention, and FIG. 10 is a schematic top view showing the second example of the endoscope image pickup device of the embodiment of the present invention. FIG. 11 is a schematic enlarged side view showing a part of the second example of the endoscope image pickup device of the embodiment of the present invention.

FIG. 9 shows a state in which a connecting member 40 is removed in the endoscope image pickup device 20a of FIG. 7.

In FIGS. 7 to 11, the same constituents as those of the endoscope image pickup device 20 shown in FIGS. 2 to 6 are designated by the same reference numerals, and detailed descriptions thereof will be omitted.

An endoscope image pickup device 20a shown in FIG. 7 has the same configurations as those of the endoscope image pickup device 20 shown in FIG. 2 except that the holder 24 has a different configuration, the connecting member 40 has a different configuration, the electronic component 30 is not disposed on a front surface 26i of the third plane portion 26e, and the protective tube 37 is not provided, as compared with the endoscope image pickup device 20 shown in FIG. 2.

In the endoscope image pickup device 20a shown in FIG. 7, the image pickup element 25 and the electronic components 30 and 30a are mounted on the circuit board 26 in the same manner as in the endoscope image pickup device 20 shown in FIG. 2.

As shown in FIG. 9, the circuit board 26 has at least the first plane portion 26a, the second plane portion 26c connected by the first plane portion 26a and the first bent portion 26b, and the third plane portion 26e connected by the second plane portion 26c and the second bent portion 26d.

The image pickup element 25 is mounted on the front surface 26f of the first plane portion 26a, and the electronic components 30 and 30a are mounted on the back surface 26g of the second plane portion 26c facing the front surface 26f of the first plane portion 26a. The electronic component 30 is mounted on the front surface 26i of the third plane portion 26e. As described above, the connection terminals (not shown) are provided on the back surface 26h of the third plane portion 26e facing the second plane portion 26c.

The circuit board 26 having the above-described configuration of the endoscope image pickup device 20a makes it possible to reduce the height of the endoscope image pickup device 20a in the Z direction and to miniaturize the endoscope image pickup device 20a. Moreover, as will be described later, a space of the endoscope image pickup device 20a can be effectively utilized.

The dispositions of the image pickup element 25, the electronic components 30 and 30a, the connection terminals, and the like on the circuit board 26 are not particularly limited.

The holder 24 has a polygonal flange portion 24b provided on an edge surface on the proximal end side of an attachment tubular portion 24a. A restricting member 24h is provided at each of both ends of the flange portion 24b in the Y direction. The restricting member 24h is provided on the side surface of the holder 24. The restricting member 24h is, for example, a projecting member, and the restricting member 24h is also an engaging projecting portion 24i. The restricting members 24h of the holder 24, that is, the engaging projecting portions 24i, are disposed so as to face each other in a disposition direction of arm portions 40c. The above-described disposition direction is the Y direction in FIG. 7. As will be described later, the arm portion 40c of the connecting member 40 is engaged with the restricting member 24h, that is, the engaging projecting portion 24i. The outer shape of the engaging projecting portion 24i is a polygonal shape. The engaging projecting portion 24i in FIG. 7 is a quadrangle, and to be exact, has an octagonal shape in which all the corners of the quadrangle are cut off by a triangle.

The prism 27 is disposed between the restricting members 24h, and the incident surface 27a is in contact with the flange portion 24b in a state of being sandwiched between the restricting members 24h. With this, the prism 27 is positioned in the X direction. The holder 24 holds the lens barrel 22 and the prism 27 at predetermined positions so that positions of the lens barrel 22 and of the prism 27 relative to each other, that is, positions of the lens barrel 22 and of the light-receiving surface 25a of the image pickup element 25 relative to each other are fixed. The emission surface 27b of the prism 27 and the image pickup element 25 face each other.

The connecting member 40 connects the holder 24 and the signal cable 28 to each other in the same manner as in the connecting member 29 shown in FIG. 2. The connecting member 40 holds the signal cable 28 therein. For example, as shown in FIG. 8, the connecting member 40 is a member formed by bending one plate material. Specifically, the connecting member 40 has a shape in which one plate material extends in the direction of the optical axis C and is bent at two bending portions. Therefore, the connecting member 40 has a substantially C shape in a cross-section perpendicular to the direction of the optical axis C.

As shown in FIG. 8, for example, the connecting member 40 has a flat plate-shaped bottom portion 40a and flat plate-shaped holding portions 40b continuous with the bottom portion 40a, which are formed by bending one plate material. In the connecting member 40, a holding portion 40b side is denoted by a proximal end 41a. The signal cable 28 is held on an inner side of the holding portion 40b.

The arm portion 40c is provided on each of the flat plate-shaped holding portions 40b facing each other with an opening in the holding portions 40b. The connecting member 40 has a pair of the arm portions 40c. The arm portion 40c is bent outward of the holding portion 40b on a proximal end 41a side and then extends linearly. Therefore, an interval between the pair of arm portions 40c is wider at a distal end 41b than at the proximal end 41a, and the interval is appropriately determined in conformity with the restricting members 24h of the holder 24 shown in FIG. 7. In addition, each of the arm portions 40c has an opening portion 40d provided at the distal end 41b.

The opening portion 40d of the arm portion 40c is engaged with the restricting member 24h (engaging projecting portion 24i) of the holder 24. The opening portion 40d surrounds at least three side surfaces of the restricting member 24h (engaging projecting portion 24i). The opening portion 40d is formed by, for example, cutting off a part of the arm portion 40c into a quadrangular shape.

The opening portion 40d may have the same shape as the outer shape of the restricting member 24h (engaging projecting portion 24i). Here, the above-described shape of the opening portion 40d being the same shape as the outer shape of the restricting member 24h (engaging projecting portion 24i) includes an error range generally allowed in the relevant technical field. Therefore, the gap between the opening portion 40d and the restricting member 24h (engaging projecting portion 24i) may not be uniform, and the size of the gap may be partially different.

In the following description, “same shape” includes an error range generally allowed in the relevant technical field as described above.

In addition, each of the arm portions 40c has, for example, an edge 40e parallel to the second plane portion 26c of the circuit board 26. The edge 40e is located above the second plane portion 26c in the Z direction, and the second plane portion 26c of the circuit board 26 is exposed in a case in which the circuit board 26 is covered with the connecting member 40 from above.

Further, for example, a cover portion 40f is provided on each of the arm portions 40c. As shown in FIG. 10, the cover portions 40f are not connected to each other in the Y direction and have a gap 40g therebetween. Further, the cover portion 40f is partially provided in the X direction, and an opening portion 40h is provided on the proximal end 41a side of the cover portion 40f Further, the cover portion 40f is a member disposed over the front surface 26i of the third plane portion 26e of the circuit board 26. Since the cover portions 40f have the gap 40g and the opening portion 40h, contact with the electronic component 30 disposed on the front surface 26i of the third plane portion 26e is avoided.

The connecting member 40 covers a part of the third plane portion 26e or the like of the circuit board 26, the prism 27, and the distal end portion of the signal cable 28, and also serves as a cover member for the circuit board 26, the prism 27, and the signal cable 28. Further, the connecting member 40 also functions as a protective member for the circuit board 26, the prism 27, and the signal cable 28.

As described above, an engaging portion 41 that engages the opening portions 40d of the pair of arm portions 40c and the restricting members 24h of the holder 24, that is, the engaging projecting portions 24i with each other is provided.

As described above, the opening portion 40d surrounds at least three side surfaces of the restricting member 24h (engaging projecting portion 24i). As shown in FIG. 11, the opening portion 40d may be configured to surround the entire outer surfaces of the restricting member 24h (engaging projecting portion 24i). For example, there is a gap 77a between one side surface 24j out of the outer surfaces of the restricting member 24h (engaging projecting portion 24i) and an inner peripheral surface 40j of the opening portion 40d. In addition, there is a gap 77b between the other side surface 24k out of the outer surfaces of the restricting member 24h (engaging projecting portion 24i) and the inner peripheral surface 40j of the opening portion 40d. The adhesive layer 74 is provided in the gaps 77a and 77b.

The adhesive layer 74 is formed by applying a filler-containing adhesive to the restricting member 24h and by engaging the opening portion 40d of the arm portion 40c and the restricting member 24h of the holder 24, that is, the engaging projecting portion 24i with each other, and then curing the filler-containing adhesive.

It is preferable that a width d₄ of the gap 77a and a width d₅ of the gap 77b are each wider than the average particle diameter of the filler contained in the adhesive layer 74. With this, for example, the thickness of the adhesive layer 74 can be maintained while leaving the filler of the filler-containing adhesive. In addition, the width d₄ of the gap 77a and the width d₅ of the gap 77b are each preferably 5 μm or more as in the first gap 70 of FIG. 5 described above. With this, the adhesive layer 74 can secure a sufficient thickness for bonding and a sufficient bonding strength is obtained. An upper limit of each of the width d₄ of the gap 77a and the width d₅ of the gap 77b is about 100 to 120 μm and more preferably 30 to 70 μm, as in the first gap 70 of FIG. 5 described above, from the viewpoint of securing the thickness of the adhesive layer 74. In this case, the upper limit of the thickness of the adhesive layer 74 is about 100 to 120 μm and more preferably 30 to 70 μm as described above from the fact that the variation in the bonding strength can be further reduced.

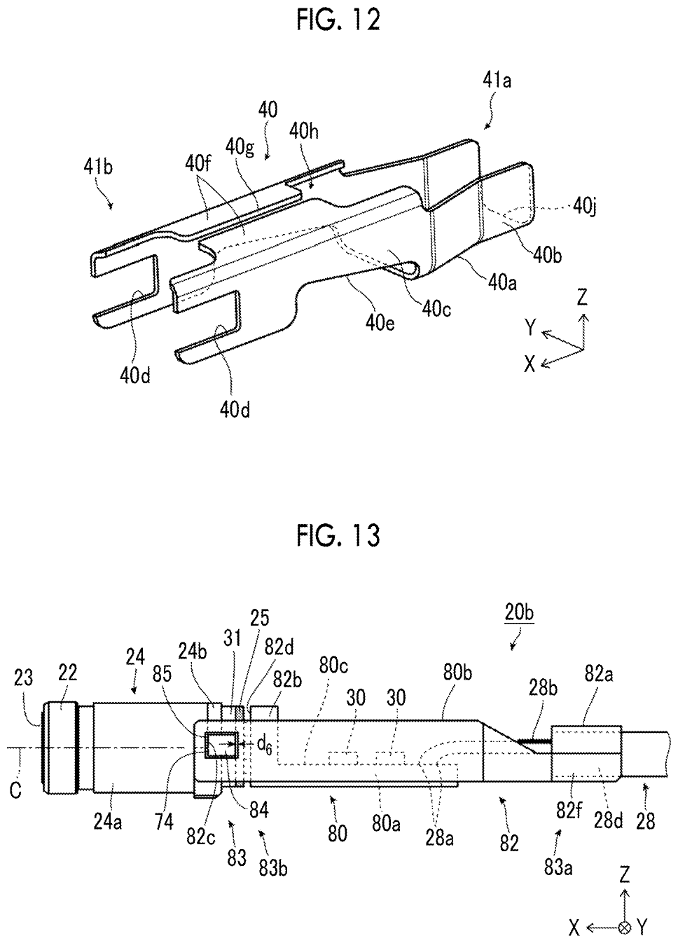

The shape of the opening portion 40d of the arm portion 40c is not limited to the quadrangle as shown in FIGS. 8 and 11, and may be a groove as in the opening portion 40d shown in FIG. 12. In this case, the opening portion 40d surrounds three side surfaces of the restricting member 24h (engaging projecting portion 24i).

The opening portion 40d of the groove is not particularly limited as long as the opening portion 40h surrounds three side surfaces of the restricting member 24h (engaging projecting portion 24i), and the shape is appropriately set in conformity with the three side surfaces of the outer shape of the restricting member 24h (engaging projecting portion 24i)

In a case in which the opening portion 40d of the arm portion 40c and the engaging projecting portion 24i of the holder 24 are engaged with each other, the adhesive layer 74 is formed by using a filler-containing adhesive, and an adhesive layer (not shown) is also formed between a side surface 24p of the holder 24 and the arm portion 40c. A thickness of this adhesive layer is the same as the thickness of the adhesive layer 74 provided in the width d₁ of the first gap 70 shown in FIG. 5 described above.

In the measurement of each of the width d₄ of the gap 77a and the width d₅ of the gap 77b, an image is acquired from an arm portion 40c side of the endoscope image pickup device 20a. The acquired image is used to specify a corresponding part corresponding to each of the width d₄ of the gap 77a and the width d₅ of the gap 77b. A length is measured using a ruler by, for example, enlarging an image of the corresponding part. The measured values are the width d₄ of the gap 77a and the width d₅ of the gap 77b.

The above-described measurement of each of the width d₄ of the gap 77a and the width d₅ of the gap 77b also include entering the acquired image of the endoscope image pickup device 20a into the computer and using software to measure the length of the corresponding part corresponding to each of the width d₄ of the gap 77a and the width d₅ of the gap 77b.

As described above, the adhesive layer 74 is used, so that it is possible to realize a firm fixation and a stable connection strength as in the above-described endoscope image pickup device 20, even with the endoscope image pickup device 20a.

In addition, as described above, since the opening portions 40d are fitted to the projecting restricting members 24h (engaging projecting portions 24i) because of the configuration in which the engaging portion 41 that engages the opening portions 40d of the pair of arm portions 40c and the restricting members 24h (engaging projecting portions 24i) of the holder 24 with each other is provided, a length of the endoscope image pickup device 20a in the Y direction orthogonal to the optical axis C can be shortened, and an increase in size of the endoscope image pickup device 20a can be suppressed. Moreover, it is possible to realize a firm fixation between the holder 24 and the connecting member 40.

A thickness of the arm portion 40c matches a height of the restricting member 24h (engaging projecting portion 24i), whereby the length of the endoscope image pickup device 20a in the Y direction orthogonal to the optical axis C can be shortened in a case in which the opening portions 40d of the pair of arm portions 40c are engaged with the restricting members 24h (engaging projecting portions 24i) of the holder 24, respectively, and with this configuration, the endoscope image pickup device 20a can be further miniaturized.

In the connecting member 40, it is preferable that the pair of arm portions 40c are bent so as to be closer to each other at the distal end 41b than at the proximal end 41a of the arm portions 40c. That is, it is preferable that the pair of arm portions 40c are bent in the closing direction. With this, the arm portions 40c are spread once, whereby the opening portions 40d of the arm portions 40c and the restricting members 24h (engaging projecting portions 24i) of the holder 24 can be fitted to each other and can be easily assembled.

As described above, it is preferable that the pair of arm portions 40c are bent so as to be closer to each other at the distal end 41b than at the proximal end 41a of the arm portions 40c, but this may be a state of components before assembly.

Further, although a configuration is employed in which the arm portion 40c is provided with the penetrating opening portion 40d or the opening portion 40h of the groove, the present invention is not limited thereto, and a bottomed recessed portion having only a recess without penetrating may be used. In this case, the adhesive layer is provided around the engaging projecting portion 24i and between the side surface 24p of the holder 24 and the arm portion 40c.

The signal cable 28 is attached to and held on the inner side of the holding portion 40b of the connecting member 40. The method of attaching the signal cable 28 is not particularly limited as long as, for example, the signal cable 28 does not come off from the holding portion 40b and the signal lines 28a do not come off during the use of the endoscope, and the signal cable 28 is attached to the connecting member 40 by using, for example, an adhesive.

In the holder 24, the two restricting members 24h (engaging projecting portions 24i) have the same size and shape, that is, are congruent, but the size and the shape may be different.

Further, in the holder 24, the shape of the outer shape of the restricting member 24h (engaging projecting portion 24i) is not particularly limited to the quadrangle, and may be a circle, an ellipse, or a polygon, such as a triangle, a pentagon, or a hexagon, or a shape formed by combining these shapes may be used. Furthermore, not only one shape but also a plurality of shapes having the same shape may be disposed or a specific pattern may be used, but it is preferable to have at least three side surfaces.

In the engaging portion 41, although one projecting portion and one recessed portion are engaged in one part, the engaging part is not limited to one, and a configuration may be employed in which one projecting portion has a plurality of engaging parts.

The size of the projecting portion of the holder 24 is preferably, for example, a size that covers at least a part of a side surface 27d of the prism 27. The size of the projecting portion is set to the size that covers at least a part of the side surface 27d of the prism 27, so that the prism 27 can be more stably sandwiched and fixed, and stable position restriction can be performed. In addition, this can be used for positioning the prism in the Y direction with respect to the holder during assembly.

An upper limit of the size of the projecting portion of the holder 24 can be a size that covers the entire side surface 27d of the prism 27.

Further, in the holder 24, the two restricting members 24h (engaging projecting portions 24i) are provided so as to face each other, so that the prism 27 and the circuit board 26 are surrounded by the arm portions 40c. With this, the holder 24 and the connecting member 40 are stably engaged with each other, and the prism 27 and the circuit board 26 can also be protected.

In the holder 24, although a configuration is employed in which the two restricting members 24h (engaging projecting portions 24i) are provided, the present invention is not limited thereto as long as the size is not increased, and three or more projecting portions may be provided. That is, the number of engaging portions can also be three or more.

In addition, the connecting member 40 is connected to each of the holder 24 and the signal cable 28 so that the connection between the connection terminals and the signal lines 28a is prevented from being disconnected because the connection part between the connection terminals provided on the circuit board 26 and the signal lines 28a of the signal cable 28 is pulled, for example, in a case in which the signal cable 28 is pulled.

A metal material for forming the connecting member 40 is not particularly limited, but a metal material having a high thermal conductivity is preferable. In consideration of workability, availability, strength, and the like, the connecting member 40 is preferably a stainless steel or a copper alloy.

The arm portion 40c of the connecting member 40 and the restricting member 24h of the holder 24, and the holding portion 40b of the connecting member 40 and the outer covering 28d of the signal cable 28 are each bonded and fixed to each other by using, for example, an adhesive. In a case of being bonded and fixed to each other, the adhesive is in a cured state. The present invention is not limited to the bonding and fixing using the adhesive as long as the holding portion 40b of the connecting member 40 and the outer covering 28d of the signal cable 28 can be fixed. As the adhesive, an epoxy resin-based adhesive, a silicone-based adhesive, or an acrylic adhesive can be used as in the above-described adhesive 38.

Further, in the endoscope image pickup device 20a, the fixing member 35 may also be provided on the outer covering 28d of the signal cable 28. The fixing member 35 is provided on the outer covering 28d of the signal cable 28, and is tightened to fix the outer covering 28d to the signal lines 28a of the signal cable 28. The outer covering 28d of the signal cable 28 is fixed with the fixing member 35, so that the fixing strength of the outer covering 28d of the signal cable 28 can be increased.

In addition, the outer covering 28d (see FIG. 9) of the signal cable 28 may be covered with the protective tube 37 (see FIG. 3), thereby protecting the signal cable 28. With this, the signal cable 28 is prevented from breaking, and the signal lines 28a are prevented from being disconnected.

In the endoscope image pickup device 20a, an observational image captured by the image pickup element 25 from the image pickup lens 23 is formed on the light-receiving surface 25a of the image pickup element 25 and converted into an electrical signal, and this electrical signal is output to the processor device 16 (see FIG. 1) via the signal cable 28 and is converted into a video signal, and an observation image is displayed on the monitor connected to the processor device 16.

In the endoscope image pickup device 20a, it is preferable that a part of the inclined surface 27c of the prism 27 and a part of the second bent portion 26d of the circuit board 26 are connected to each other by a photocurable adhesive (not shown), and that a part of the first bent portion 26b and/or a part of the second plane portion 26c, and a part of the signal cable 28 and/or the third plane portion 26e are connected to each other by a photocurable adhesive (not shown). With this, the shape of the circuit board 26 can be maintained, and the manufacturing time of the endoscope image pickup device 20a can be shortened. As the photocurable adhesive, the above-described adhesive can be used.

### Third Example of Endoscope Image Pickup Device

FIG. 13 is a schematic side view showing a third example of the endoscope image pickup device of the embodiment of the present invention.

In FIG. 13, the same constituents as those of the endoscope image pickup device 20 shown in FIGS. 2 to 6 are designated by the same reference numerals, and detailed descriptions thereof will be omitted.

An endoscope image pickup device 20b shown in FIG. 13 is different from the endoscope image pickup device 20 shown in FIGS. 2 to 6 in that the configuration of the holder 24, the configuration of a circuit board 80, and the dispositions of the image pickup element 25 and of the cover glass 31 are different, and the prism 27 (see FIG. 3) is not provided. Other configurations are the same as those of the endoscope image pickup device 20 shown in FIGS. 2 to 6.

In the endoscope image pickup device 20b shown in FIG. 13, engaging projecting portions 84 are provided at both ends of the flange portion 24b of the holder 24 in the Y direction. The engaging projecting portion 84 is provided on the side surface of the holder 24.

In addition, the circuit board 80 is provided with a support portion 80b perpendicular to a flat plate-shaped base portion 80a. The image pickup element 25 is provided on a support surface 80d of the support portion 80b. The cover glass 31 is provided on the image pickup element 25, and a light-receiving surface (not shown) is covered with the cover glass 31.

The cover glass 31 is disposed on a back surface of the flange portion 24b of the holder 24, and the image pickup element 25 is disposed with the light-receiving surface (not shown) facing the image pickup lens 23.

A front surface 80c of the circuit board 80 is provided with a plurality of connection terminals (not shown) through which signals or power is input and output to and from the image pickup element 25 and the electronic component 30. The signal lines 28a of the signal cable 28 are electrically connected to the connection terminals of the front surface 80c of the circuit board 80.

The configuration of the circuit board 80 is not particularly limited, and may be a flexible wiring board or a rigid board. A printed wiring board can be used as the rigid board.

As shown in FIG. 13, a connecting member 82 has an arc formed by bending one plate material, and holding portions 82a each having a flat plate-shaped base material portion 82f continuous with the arc. In the connecting member 82, a holding portion 82a side is denoted by a proximal end 83a. In a case in which the base material portion 82f is squeezed so as to press the signal cable 28, the base material portion 82f is bent along the outer covering 28d of the signal cable 28.

An arm portion 82b is provided on each of the flat plate-shaped base material portions 82f facing each other with an opening in the holding portions 82a. The connecting member 82 has a pair of the arm portions 82b. The arm portion 82b is bent outward of the holding portion 82a on a proximal end 83a side and then extends linearly. Therefore, an interval between the pair of arm portions 82b is wider at a distal end 83b than at the proximal end 83a, and the interval is appropriately determined in conformity with the engaging projecting portions 84 of the holder 24. In addition, each of the arm portions 82b has an opening portion 82c provided at the distal end 83b.

The opening portion 82c of the arm portion 82b is engaged with the engaging projecting portion 84 of the holder 24. For example, a shape of the opening portion 82c is the same shape as an outer shape of the engaging projecting portion 84. As described above, an engaging portion 83 that engages the opening portions 82c of the pair of arm portions 82b and the engaging projecting portions 84 of the holder 24 with each other is provided.

Here, the above-described shape of the opening portion 82c being the same shape as the outer shape of the engaging projecting portion 84 includes an error range generally allowed in the relevant technical field. Therefore, a gap between the opening portion 82c and the engaging projecting portion 84 may not be uniform, and the size of the gap may be partially different.

The opening portion 82c is engaged with the engaging projecting portion 84 and surrounds at least three side surfaces of the engaging projecting portion 84. In FIG. 13, the opening portion 82c surrounds all the side surfaces of the engaging projecting portion 84. As shown in FIG. 12, the opening portion 82c may be a groove that surrounds three side surfaces of the engaging projecting portion 84.

There is a gap 85 between the engaging projecting portion 84 and the opening portion 82c. The adhesive layer 74 is provided in the gap 85.

The shape of the opening portion 82c may be the same as that of the opening portion 40d of the above-described endoscope image pickup device 20a. A shape of the engaging projecting portion 84 may be the same as that of the engaging projecting portion 24i of the above-described endoscope image pickup device 20a.

It is preferable that a width d₆ of the gap 85 is wider than the average particle diameter of the filler contained in the adhesive layer 74, as in the configuration shown in FIG. 11. Further, the width d₆ of the gap 85 is preferably 5 μm or more. An upper limit of the width d₆ of the gap 85 is about 100 to 120 μm and more preferably 30 to 70 μm, as in the first gap 70 of FIG. 5 described above, from the viewpoint of securing the thickness of the adhesive layer 74. In this case, the upper limit of the thickness of the adhesive layer 74 is about 100 to 120 μm and more preferably 30 to 70 μm as described above from the fact that the variation in the bonding strength can be further reduced.

In a case in which the gap between the engaging projecting portion 84 and the opening portion 82c is not uniform, a narrowest part is preferably wider than the average particle diameter of the filler contained in the adhesive layer 74 and is preferably 5 μm or more.

In the measurement of the width d₆ of the gap 85, an image from an arm portion 82b side of the endoscope image pickup device 20b is acquired. The acquired image is used to specify a corresponding part corresponding to the width d₆ of the gap 85. A length is measured using a ruler by, for example, enlarging an image of the corresponding part. The measured value is the width d₆ of the gap 85. The above-described measurement of the width d₆ of the gap 85 also includes entering the acquired image of the endoscope image pickup device 20b into the computer and using software to measure the length of the corresponding part corresponding to the width d₆ of the gap 85.

The endoscope image pickup device 20b shown in FIG. 13 can also obtain the same effect as that of the endoscope image pickup device 20. Even in the endoscope image pickup device 20b, it is possible to realize a firm fixation and a stable connection strength.

Similarly to the endoscope image pickup device 20, the endoscope image pickup device 20b may also be provided with the fixing member 35 (see FIG. 3) on the signal cable 28 to increase the fixing strength of the outer covering 28d of the signal cable 28. Further, the signal cable 28 may be covered with the protective tube 37 (see FIG. 3), thereby protecting the signal cable 28. With this, the signal cable 28 is prevented from breaking, and the signal lines 28a are prevented from being disconnected.

### Adhesive Layer

The adhesive layer provided for engagement between the holder and the connecting member and containing a filler for bonding the holder and the connecting member has the filler and a resin containing a filler. The adhesive layer is formed of, for example, a filler-containing adhesive, and is formed by curing the filler-containing adhesive. The adhesive layer is, for example, an adhesive layer in a state in which the filler-containing adhesive is cured.

As described above, from the viewpoint of securing the thickness of the adhesive layer and reducing the variation in the bonding strength, the adhesive layer preferably has a thickness of 5 μm or more, and the upper limit thereof is about 100 to 120 μm and more preferably 30 to 70 μm from the fact that the variation in the bonding strength can be further reduced.

The filler of the adhesive layer is for maintaining the thickness of the adhesive layer. The form of the filler is particles, a disc, a needle, or the like, and is not particularly limited. The filler has, for example, a form of particles, and beads are used.

In a case in which the filler has the form of particles, the average particle diameter is preferably 5 μm or more from the viewpoint of maintaining the thickness of the adhesive layer. This average particle diameter is a value measured by a specific surface area measuring method (BET method).

It is preferable that the filler has the form of particles because, in a case in which the adhesive layer is provided in the gap, the gap is easily filled with the filler and the thickness of the adhesive layer can be easily controlled.

The resin is formed of an epoxy-based adhesive, a silicone-based adhesive, or an acrylic adhesive.

The filler is not particularly limited, and for example, aluminum nitride, alumina, magnesium oxide, or hexagonal boron nitride is used.

Examples of the filler-containing adhesive include a filler-containing adhesive obtained by mixing a filler with an adhesive, such as an epoxy-based adhesive, a silicone-based adhesive, or an acrylic adhesive. The filler-containing adhesive may be a filler-containing adhesive in which a filler is mixed with an adhesive in advance, or a filler-containing adhesive may be obtained by mixing a filler with an adhesive. As described above, the average particle diameter of the filler is preferably 5 μm or more, and in the filler-containing adhesive, for example, alumina beads having an average particle diameter of 20 μm are mixed as the filler.

In the adhesive layer, it is preferable that the thermal conductivity of the filler is higher than the thermal conductivity of the resin. With this, thermal conduction between the holder and the connecting member is improved, and heat from the electronic component or the like can be efficiently conducted to the outside of the endoscope image pickup device. The thermal conductivity of the filler is a catalog value in a case in which the filler is a commercially available product. Further, regarding the resin, in a case in which the adhesive constituting the resin is a commercially available product, the thermal conductivity is a catalog value.

The present invention is basically configured as described above. Although the endoscope image pickup device and the endoscope of the embodiment of the present invention have been described in detail above, the present invention is not limited to the above-described embodiment, and needless to say, various modifications or changes may be made without departing from the gist of the present invention.

### EXPLANATION OF REFERENCES

10: endoscope system
12: endoscope
14: light source device
16: processor device
20, 20a, 20b: endoscope image pickup device
22: lens barrel
23: image pickup lens
24: holder
24a: attachment tubular portion
24b: flange portion
24c, 24h: restricting member
24d: projecting portion
24e: projecting portion
24f: groove
24j, 84: engaging projecting portion
24j, 24k: side surface
24m, 24p: side surface
25: image pickup element
25a: light-receiving surface
26, 80: circuit board
26a: first plane portion
26b: first bent portion
26c: second plane portion
26d: second bent portion
26e: third plane portion
26f: front surface
26g, 26h: back surface
27: prism
27a: incident surface
27b: emission surface
27c: inclined surface
27d: side surface
27e: reflecting surface
28: signal cable
28a: signal line
28b: covering layer
28c: shielded conductor
28d: outer covering
29, 40, 82: connecting member
29a, 40c, 82b: arm portion
29b, 80a: base portion
29c: side surface portion
29d, 82a: holding portion
29e: rear edge surface
29f: recessed portion
29g: edge
29h: distal end portion
29i: back surface
29j: upper edge
29k: lower edge
29n: latch portion
29p: inner edge
30, 30a: electronic component
31: cover glass
34: bump
35: fixing member
35b: end
37: protective tube
37a: distal end
38: adhesive
39: gap
40a: bottom portion
40b: holding portion
40d, 40h, 82c: opening portion
40e: edge
40f: cover portion
40g: gap
40j: inner peripheral surface
41, 76, 83: engaging portion
41a, 83a: proximal end
41b, 83b: distal end
70: first gap
71: second gap
72: third gap
74, 75: adhesive layer
77a, 77b: gap
80b: support portion
80c: front surface
80d: support surface
82f: base material portion
85: gap
C: optical axis
d₁, d₂, d₃, d₄, d₅, d₆: width
h₁, h₂: height

What is claimed is:

1. An endoscope image pickup device that acquires an image of an observation target, the endoscope image pickup device comprising:
a lens barrel provided with an internal image pickup lens;
an image pickup element that receives light which has passed through the image pickup lens to perform photoelectric conversion;
a holder that holds the lens barrel;
a signal cable electrically connected to the image pickup element; and
a connecting member that connects the holder and the signal cable to each other,
wherein the holder and the connecting member are engaged with each other at an engaging portion, and the engaging portion is provided with an adhesive layer containing a filler.

2. The endoscope image pickup device according to claim 1,
wherein the adhesive layer has a thickness of 5 μm or more.

3. The endoscope image pickup device according to claim 1,
wherein the connecting member has a pair of arm portions facing each other,
the holder has a projecting portion on a side surface, and
the projecting portion protrudes in a direction perpendicular to the side surface with respect to the arm portion in a state in which the arm portion is bonded to the side surface of the holder via the adhesive layer.

4. The endoscope image pickup device according to claim 3,
wherein the filler has a form of particles, and
a height of the projecting portion is larger than an average particle diameter of the filler.

5. The endoscope image pickup device according to claim 3,
wherein the filler has a form of particles,
the projecting portions are disposed on the side surface of the holder so as to face each other at an interval along a height direction orthogonal to an optical axis of the image pickup lens,
the arm portion is disposed between the projecting portions, and
a gap between the projecting portion and the arm portion in the height direction is wider than an average particle diameter of the filler.

6. The endoscope image pickup device according to claim 3,
wherein the arm portion has a latch portion that latches onto the projecting portion of the holder in an optical axis direction of an optical axis of the image pickup lens, and
a position of the connecting member in the optical axis direction is restricted by the latch portion.

7. The endoscope image pickup device according to claim 3,
wherein a distal end portion of each of the pair of arm portions is bent inward,
the holder has a groove that is engaged with the distal end portion of the arm portion, and
the groove is provided with the adhesive layer in a state in which the distal end portion is locked to the groove.

8. The endoscope image pickup device according to claim 1,
wherein the connecting member has a pair of arm portions facing each other and has an opening portion provided at a distal end of the arm portion,
the holder has engaging projecting portions disposed so as to face each other in a disposition direction of the arm portions, and the engaging projecting portion has a polygonal outer shape,
the opening portion of the arm portion is engaged with the engaging projecting portion of the holder, and the opening portion surrounds at least three side surfaces of the engaging projecting portion, and
there is a gap between the engaging projecting portion and the opening portion, the filler has a form of particles, and the gap is wider than an average particle diameter of the filler.

9. The endoscope image pickup device according to claim 1,
wherein the adhesive layer has the filler and a resin containing the filler, and
a thermal conductivity of the filler is higher than a thermal conductivity of the resin.

10. The endoscope image pickup device according to claim 2,
wherein the connecting member has a pair of arm portions facing each other,
the holder has a projecting portion on a side surface, and
the projecting portion protrudes in a direction perpendicular to the side surface with respect to the arm portion in a state in which the arm portion is bonded to the side surface of the holder via the adhesive layer.

11. The endoscope image pickup device according to claim 4,
wherein the arm portion has a latch portion that latches onto the projecting portion of the holder in an optical axis direction of an optical axis of the image pickup lens, and
a position of the connecting member in the optical axis direction is restricted by the latch portion.

12. The endoscope image pickup device according to claim 5,
wherein the arm portion has a latch portion that latches onto the projecting portion of the holder in an optical axis direction of an optical axis of the image pickup lens, and
a position of the connecting member in the optical axis direction is restricted by the latch portion.

13. The endoscope image pickup device according to claim 4,
wherein a distal end portion of each of the pair of arm portions is bent inward,
the holder has a groove that is engaged with the distal end portion of the arm portion, and
the groove is provided with the adhesive layer in a state in which the distal end portion is locked to the groove.

14. The endoscope image pickup device according to claim 5,
wherein a distal end portion of each of the pair of arm portions is bent inward,
the holder has a groove that is engaged with the distal end portion of the arm portion, and
the groove is provided with the adhesive layer in a state in which the distal end portion is locked to the groove.

15. The endoscope image pickup device according to claim 2,
wherein the connecting member has a pair of arm portions facing each other and has an opening portion provided at a distal end of the arm portion,
the holder has engaging projecting portions disposed so as to face each other in a disposition direction of the arm portions, and the engaging projecting portion has a polygonal outer shape,
the opening portion of the arm portion is engaged with the engaging projecting portion of the holder, and the opening portion surrounds at least three side surfaces of the engaging projecting portion, and
there is a gap between the engaging projecting portion and the opening portion, the filler has a form of particles, and the gap is wider than an average particle diameter of the filler.

16. The endoscope image pickup device according to claim 2,
wherein the adhesive layer has the filler and a resin containing the filler, and
a thermal conductivity of the filler is higher than a thermal conductivity of the resin.

17. The endoscope image pickup device according to claim 3,
wherein the adhesive layer has the filler and a resin containing the filler, and
a thermal conductivity of the filler is higher than a thermal conductivity of the resin.

18. An endoscope comprising:
the endoscope image pickup device according to claim 1.

19. An endoscope comprising:
the endoscope image pickup device according to claim 2.

20. An endoscope comprising:
the endoscope image pickup device according to claim 3.
